(19) United States
(12) Patent Application Publication
(10) Pub. No.: US 2016/0213868 A1
(43) Pub. Date: Jul. 28, 2016

(54) RESPIRATORY THERAPY APPARATUS AND METHODS

(71) Applicant: SMITHS MEDICAL INTERNATIONAL LIMITED, Kent (GB)

(72) Inventors: Mohammad Qassim Mohammad Khasawneh, Luton (GB); Jeevan Sagoo, Bedfordshire (GB); Mark Sinclair Varney, Bedfordshire (GB)

(73) Assignee: SMITHS MEDICAL INTERNATIONAL LIMITED, Ashford, Kent TN25 4BF (GB)

(21) Appl. No.: 14/916,254

(22) PCT Filed: Jul. 31, 2014
(86) PCT No.: PCT/GB2014/000298

§ 371 Date: Mar. 3, 2016

(30) Foreign Application Priority Data
Sep. 12, 2013 (GB) 1316223.5

(51) Int. Cl. A61M 16/00; A61B 5/00

(52) U.S. Cl. CPC A61M 16/0006; A61B 5/4836; A61M 2230/40; A61M 2230/005; A61M 2205/583; A61M 2205/581

(57) ABSTRACT

Respiratory therapy apparatus includes an expiratory therapy device (100) that produces an oscillating resistance to breathing. The device has a vibration sensor (20) mounted on its housing (2) that transmits signals to a receiver (200) indicative of operation of the device. An accelerometer (201) is secured to the chest of the patient; this also transmits signals to the receiver (200). The apparatus prompts the user to use the device (100) at each of its different settings and the receiver (300) monitors the effect of the different settings and computes which gives the most benefit. The receiver (300) then prompts the user to select the optimum set ting for further therapy.

This invention relates to respiratory therapy apparatus of the kind including a respiratory therapy device that produces an oscillating resistance to breathing through the device, the therapy device having a plurality of different operation settings.

Patients with respiratory system diseases (such as asthma, COPD, cystic fibrosis or the like) suffer from hyper-secretion of mucus as a prominent pathophysiological feature. Moreover, those patients with hyper-secretion often also have impaired mucus transport. This imbalance between mucus transport and secretion results in mucus retention in the respiratory system.

Vibratory respiratory positive expiratory pressure (V-PEP) or oscillatory PEP (OPEP) devices are modern devices for applying chest physiotherapy. These devices apply chest physiotherapy by providing an alternating resistance to flow and have been found to be particularly effective. One example of such apparatus is sold under the trade mark Acapella (a registered trade mark of Smiths Medical) by Smiths Medical and is described in U.S. Pat. No. 6,581,598, U.S. Pat. No. 6,776,159, U.S. Pat. No. 7,059,324 and U.S. Pat. No. 7,699,054. Other vibratory respiratory therapy apparatus is available, such as “Quake” manufactured by Thayer, “AeroPEP” manufactured by Monaghan, “TheraPEP” manufactured by Smiths Medical, “IPV Percussionator” manufactured by Percussionaire Corp, and the “Flutter” and “Lung Flute” devices, amongst others. These devices are used by patients who suffer from mucus hyper-secretions and retention to help them clear the secretions from their lungs. The Acapella V-PEP device combines the principles of low-frequency vibration and positive expiratory pressure by employing a counterweighted lever and magnet to produce oscillatory positive pressures during expiration. This generated vibratory positive pressure works by mechanically reducing the viscoelasticity of the sputum by breaking down the bonds of mucus macromolecules which, in turn, enhances mucociliary clearance.

More recently it has been proposed to Exidicate the effectiveness of this therapy by placing a vibration sensor on the patient's chest, as described in PCT/GB2014/00020. In PCT/GB2014/000184 it is proposed to mount a vibration sensor on the casing of the therapy device to provide a signal indicative of use of the device. In PCT/GB2014/000177 it is proposed to use an external audio sensor, such as in a mobile phone, to monitor use of the device. These more recent proposals are mainly concerned with monitoring patient compliance.

The frequency and amplitude of the generated vibration in the chest is highly dependent on the manner in which the V-PEP device is used.

Typically, clinicians or respiratory therapists are responsible for selecting the appropriate airway clearance therapy for a particular patient (“The Value of Conducting Laboratory Investigations on Airway Clearance Devices,” 2008; Respiratory Therapist Series, 1985; California Thoracic Society, 2006). They are also responsible for optimizing the operation of V-PEP devices to achieve the desired therapy goals (Hristara-Papadopoulou et al., 2008; California Thoracic Society, 2006; Myers, 2007).

It is established in the literature that expectoration is optimized when the applied frequency coincides with the ciliary movement range and with the respiratory system resonance (Gosselink, 2006; Eduardo et al., 2009; dos Santos et al., 2013b). Nonetheless, the resonant frequency of the lungs is a dynamic value that might increase or decrease depending on the level of airway obstruction and disease prognosis (de Lima et al., 2005; Cavalcanti et al., 2006; Di Mango et al., 2006). The resonant frequency will vary from patient to patient.

Clinicians and respiratory therapists need to have knowledge of airway clearance techniques, the patient cognitive ability and disease prognosis, as well as therapeutic goals, in order to be able to make a well informed decision when selecting the appropriate device and optimizing device setting (“The Value of Conducting Laboratory Investigations on Airway Clearance Devices,” 2008). Moreover, because patients suffering from mucus hyper-secretion and retention have varying degrees of flow limitation; clinicians or respiratory therapists need to understand how certain V-PEP devices perform across the spectrum of flow ranges (“The Value of Conducting Laboratory Investigations on Airway Clearance Devices,” 2008; Hristara-Papadopoulou et al., 2008).

In addition, there are no guidelines to aid clinicians and respiratory therapists in optimizing the device operation according to the specific requirements of individual patients and continuously changing variables (“The Value of Conducting Laboratory Investigations on Airway Clearance Devices,” 2008). Additionally, manufacturer's instructions for using V-PEP devices may be vague and often lack the required specifications (Eduardo et al., 2009; Antonio and Pt, 2008). In short, patients often use vibratory PEP devices without any clear idea about which setting works best for them the element of “feedback” is missing.

It is an object of the present invention to provide alternative respiratory therapy apparatus and methods.

According to one aspect of the present invention there is provided respiratory therapy apparatus of the above-specified kind, characterised in that the apparatus includes a first sensor associated with the device arranged to provide a first signal dependent on operation of the device, a second sensor arranged to produce a second signal responsive to vibration in respiratory passages of the patient caused by use of the device, and a receiver for receiving the first and second signals to provide an indication of the device setting to produce the optimum therapeutic effect on the patient.

The first sensor may include a vibration sensor and is preferably mounted on a housing of the device. The second sensor may include a vibration sensor and an arrangement for mounting the second sensor in vibration contact with the patient's chest. The second sensor may include a three-axis accelerometer. The receiver is preferably a wireless receiver. The receiver may include a display by which the indication of optimum device setting is provided. The respiratory therapy device is preferably an expiratory therapy device. The different operation settings are preferably different frequency settings. The apparatus is preferably arranged to record different optimum settings indicated by the apparatus.

According to another aspect of the present invention there is provided a method for determining the optimum setting of a respiratory therapy device of the kind having a plurality of different settings and arranged to produce an oscillating resistance to breathing through the device, characterised in that the method includes the steps of breathing through the device at a plurality of different settings, monitoring operation of the device during use at the different settings, monitoring the vibration effect in respiratory passages of the patient caused by use of the device at the different settings, and accordingly providing an indication of an optimum device setting to produce the optimum therapeutic effect on the patient.

The method preferably includes the step of prompting the user to change the setting of the device. The user may be prompted to change the setting of the device by a visual or audible cue.

According to a further aspect of the present invention there is provided apparatus for use in the method according to the above other aspect of the present invention.

Apparatus and a method for determining and selecting the optimum setting for a respiratory therapy device will now be described, by way of example, with reference to the accompanying drawings, in which:

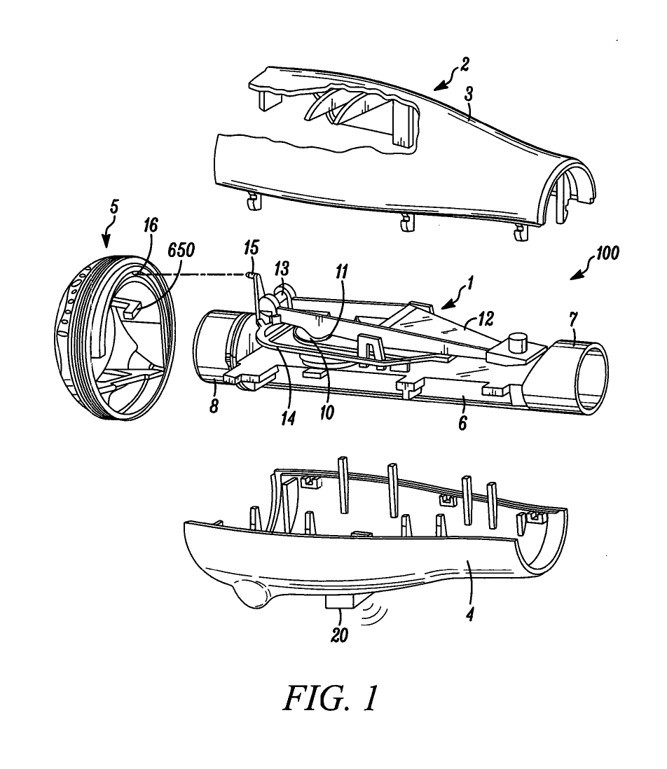

FIG. 1 is an exploded view of the respiratory therapy device; and

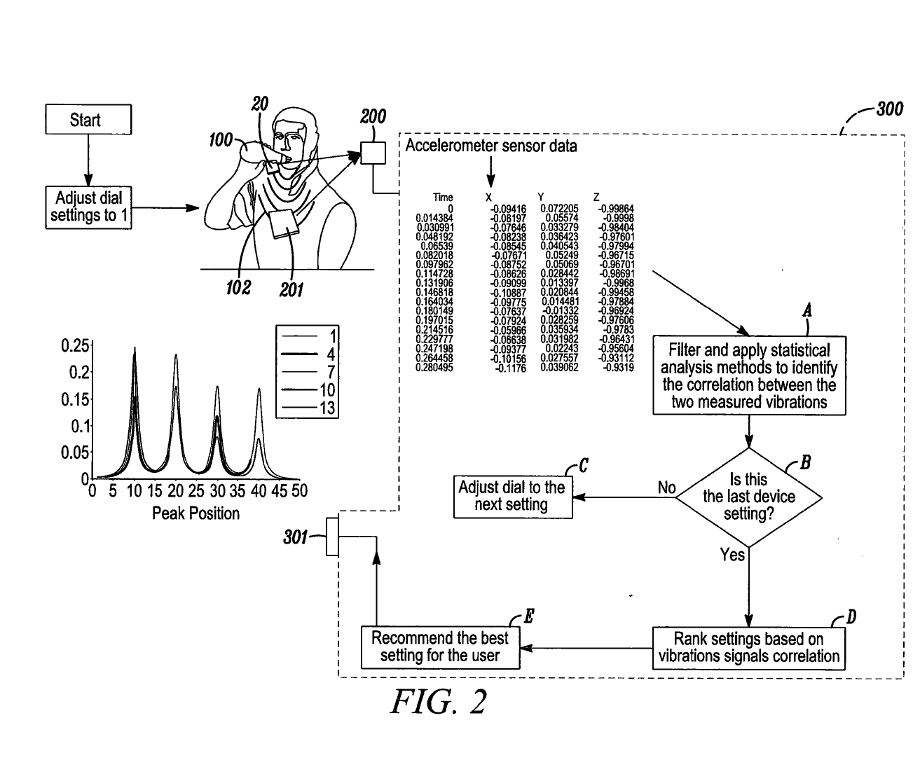

FIG. 2 illustrates the apparatus and how the device is used.

With reference first to FIG. 1, the respiratory therapy device 100 comprises a rocker assembly 1 contained within an outer housing 2 provided by an upper part 3 and a lower part 4 of substantially semi-cylindrical shape. The device is completed by an adjustable dial 5 of circular section. The rocker assembly 1 includes an air flow tube 6 with a breathing inlet 7 at one end and an inspiratory inlet 8 at the opposite end including a one-way valve (not shown) that allows air to flow into the air flow tube 6 but prevents air flowing out through the inspiratory inlet. The air flow tube 6 has an outlet opening 10 with a non-linear profile that is opened and closed by a conical valve element 11 mounted on a rocker arm 12 pivoted midway along its length about a transverse axis. The air flow tube 6 and housing 2 provide a structure with which the rocker arm 12 is mounted. At its far end, remote from the breathing inlet 7, the rocker arm 12 carries an iron pin 13 that interacts with the magnetic field produced by a permanent magnet (not visible) mounted on an adjustable support frame 14. The magnet arrangement is such that, when the patient is not breathing through the device, the far end of the rocker arm 12 is held down such that its valve element 11 is also held down in sealing engagement with the outlet opening 10. A cam follower projection 15 at one end of the support frame 14 locates in a cam slot 16 in the dial 5 such that, by rotating the dial, the support frame 14, with its magnet, can be moved up or down to alter the strength of the magnetic field interacting with the iron pin 13. The dial 5 enables the frequency of operation and the resistance to flow of air through the device to be adjusted for maximum therapeutic benefit to the user. Other V-PEP devices may have different setting arrangements for adjusting operation of the device and may be graduated in other ways, such as in frequency.

When the patient inhales through the breathing inlet 7 air is drawn through the inspiratory inlet 8 and along the air flow tube 6 to the breathing inlet. When the patient exhales, the one-way valve in the inspiratory inlet 8 closes, preventing any air flowing out along this path. Instead, the expiratory pressure is applied to the underside of the valve element 11 on the rocker arm 12 causing it to be lifted up out of the opening 10 against the magnetic attraction, thereby allowing air to flow out to atmosphere. The opening 10 has a non-linear profile, which causes the effective discharge area to increase as the far end of the rocker arm 12 lifts, thereby allowing the arm to fall back down and close the opening. As long as the user keeps applying sufficient expiratory pressure, the rocker arm 12 will rise and fall repeatedly as the opening 10 is opened and closed, causing a vibratory, alternating or oscillating interruption to expiratory breath flow through the device. Further information about the construction and operation of the device can be found in US6581598, the contents of which are hereby incorporated into the present application.

The device 100 additionally includes a vibration sensor 20 attached to the structure of the device. In particular, the sensor 20 is attached to the external surface of the housing 2. Although it would be possible to mount the sensor 20 internally within the housing 2, mounting the sensor externally avoids the need to provide electrical access within the device. The sensor 20 is responsive to vibration transmitted through the structure of the device caused by parts of the rocker arm 12 contacting other parts of the device as it oscillates up and down in see-saw fashion. The sensor 20 could be of any conventional kind responsive to vibration, such as an accelerometer provided by a MEMS structure, or a piezoelectric element, pressure sensor, SAW device or the like. The sensor 20 preferably includes a wireless transmitter, such as a Bluetooth radio frequency transmitter, which transmits vibration information to a remote receiver 200 (FIG. 2). Alternatively, the sensor could provide an output via a cable.

FIG. 2 shows a patient holding the respiratory therapy device 100 shown in FIG. 1 up to his mouth. The patient wears a second sensor 201 in the form of a three-axis accelerometer in such a way as to respond to vibrations in the chest or respiratory passages. In particular, the sensor 201 could be carried by a halter 102 around the patient's neck and shoulders. Ideally the sensor 201 is located in the middle of the sternum or on the abdomen. Alternatively, it could be carried by a chest strap or secured adhesively to the chest. The chest sensor 201 also generates signals, either wirelessly or via a cable, in response to detected vibration within the chest, which signals are supplied to the receiver 200. The receiver 200 connects with a processor 300 arranged to provide an indication of the optimum therapeutic setting of the respiratory device 100. This is achieved by the user initially setting the dial 5 to the lowest setting “1” and breathing through the device 100. The user may be prompted to do this by instruction from the processor 300. As he does this, the device sensor 20 provides an output to the receiver 200, which is indicative of the frequency and amplitude of vibration of the device 100. At the same time, the chest sensor 201 provides an output to the receiver 200 indicative of frequency and amplitude of vibration within the chest and other respiratory passages caused by use of the therapy device 100. These two signals are filtered and statistical analysis is applied to identify correlation between the two vibration signals as indicated by box “A”. The statistical techniques used could include frequency domain, such as fast Fourier transformation (FFT), time-domain (cross-correlation and auto-correlation) or other techniques. The processor 300 determines whether or not these signals originate from the last device dial setting as indicated in box “B”. Typically the device might have five settings “1” to “5” so the processor 300 simply determines whether it has received signals from all five settings. If it has not, it provides a signal to the user prompting him to turn the dial 5 to the next setting as indicated by box “C”. This may be done by an acoustic or visual cue, such as a buzzer, spoken command or by a light on the receiver 200. If step “B” indicates that the apparatus has received information from all the dial settings, the processor 300 ranks the settings based on the correlation of the vibration signals, as indicated in box “D”. From this the processor 300 recommends the best setting to achieve the optimum therapeutic effect for the particular user as indicated in box “E”. This may be done by a visual or audible cue such as by means of a visual display 301 or by an audible output, such as “Use setting 3”. Typically the recommended setting will be that at which the vibration frequency of the device 100 is matched with the vibration produced in the respiratory passages.

It is not essential for the user manually to change the setting of the device since, in other devices, this could be performed automatically by means of a motor or actuator within the device controlled by signals from the processor.

The user preferably undergoes this set up procedure when first trained to use the V-PEP device so that he subsequently uses the therapy device at the optimum setting. Periodically, however, he should repeat the set up procedure since his respiratory condition may change as a result of the treatment or because of degeneration of his condition. He can then adjust the dial setting to accommodate these changes so that he always achieves maximum benefit.

The apparatus preferably records changes in the recommended settings since this could give an indication to the clinician of changes in the patient's clinical state that might require alternative or additional treatment.

The invention is not confined to expiratory therapy apparatus but could be used with inspiratory therapy apparatus.

1-14. (canceled)

15. Respiratory therapy apparatus including a respiratory therapy device of the kind arranged to produce an oscillating resistance to breathing through the device, the therapy device having a plurality of different operation settings, characterized in that the apparatus includes a first sensor associated with the device arranged to provide a first signal dependent on operation of the device, a second sensor arranged to produce a second signal responsive to vibration in respiratory passages of the patient caused by use of the device, and a receiver for receiving the first and second signals to provide an indication of the device setting to produce the optimum therapeutic effect on the patient.

16. Apparatus according to claim 15, characterized in that the first sensor includes a vibration sensor.

17. Apparatus according to claim 16, characterized in that the vibration sensor is mounted on a housing of the device.

18. Apparatus according to claim 15, characterized in that the second sensor includes a vibration sensor and an arrangement for mounting the vibration sensor in vibration contact with the patient's chest.

19. Apparatus according to claim 18, characterized in that the vibration sensor includes a three-axis accelerometer.

20. Apparatus according to claim 15, characterized in that the receiver is a wireless receiver.

21. Apparatus according to claim 15, characterized in that the receiver includes a display by which the indication of optimum device setting is provided.

22. Apparatus according to claim 15, characterized in that the respiratory therapy device is an expiratory therapy device.

23. Apparatus according to claim 15, characterized in that the different operation settings are different frequency settings.

24. Apparatus according to claim 15, characterized in that the apparatus is arranged to record different optimum settings indicated by the apparatus.

25. A method for determining the optimum setting of a respiratory therapy device of the kind having a plurality of different settings and arranged to produce an oscillating resistance to breathing through the device, characterized in that the method includes the steps of breathing through the device at a plurality of different settings, monitoring operation of the device during use at the different settings, monitoring the vibration effect in respiratory passages of the patient caused by use of the device at the different settings, and accordingly providing an indication of an optimum device setting to produce the optimum therapeutic effect on the patient.

26. A method according to claim 25, characterized in that the method includes the step of prompting the user to change the setting of the device.

27. A method according to claim 26, characterized in that the user is prompted to change the setting of the device by a visual or audible cue.

28. A respiratory therapy device of the kind having a plurality of different settings and arranged to produce an oscillating resistance to breathing through the device, a method for determining the optimum setting of the device, characterized in that the method includes the steps of breathing through the device at the plurality of different settings, monitoring operation of the device during use at the different settings, monitoring the vibration effect in respiratory passages of the patient caused by use of the device at the different settings, and accordingly providing an indication of an optimum device setting to produce the optimum therapeutic effect on the patient.
